(19) United States
(12) Patent Application Publication
(10) Pub. No.: US 2017/0095386 A1
(43) Pub. Date: Apr. 6, 2017

(54) PORTABLE DEFECATION ASSISTIVE EQUIPMENT

(71) Applicant: Chin Chang Trading Company, Pingtung City (TW)

(72) Inventor: Mao-Chi CHENG, Pingtung City (TW)

(73) Assignee: Chin Chang Trading Company, Pingtung City (TW)

(21) Appl. No.: 14/873,670

(22) Filed: Oct. 2, 2015

(51) Int. Cl. A61G 9/00; A61M 1/00

(52) U.S. Cl. CPC A61G 9/003; A61M 1/0066

(57) ABSTRACT

The portable defecation assistive equipment comprises a collecting receptacle, a guiding cover, and a pump motor. The collecting receptacle has an opening and a handle. The guiding cover is connected to the collecting receptacle and has a guiding hole. The guiding hole communicates with the opening of the collecting receptacle. The pump motor is connected to the collecting receptacle. The portable defecation assistive equipment has a wing radially formed around the guiding cover, such that the wing and guiding cover can seal the buttock of the user to maintain the negative pressure condition and avoid the reduction of attraction force. The portable defecation assistive equipment adopts a pump motor having smaller attraction. So the portable defecation assistive equipment has no need to adopt an additional pressure regulator valve, the structure is simplified, and the risk of the breakdown of the pressure regulator valve is avoided.

### BACKGROUND OF THE INVENTION

1. Field of the Invention

The present invention relates to an equipment for assisting defecation, especially a portable defecation assistive equipment having an air evacuating function.

2. Description of the Prior Arts

In recent years, daily care problems of the elderly and disabled people have gradually emerged. Because of declined physical functions, the elderly and disabled people have difficulty defecating by themselves. Therefore, the inventor has developed a defecation assistive equipment to assist the elderly and the disabled with defecating by the way of negative pressure and non-invasive approach.

To meet different requirements, the inventor has further developed multiple types of defecation assistive equipments such as a defecation assistive equipment mounted on a toilet, a defecation assistive equipment mounted on a chair, or a portable defecation assistive equipment for mobile use when the elderly and the disabled are unable to move to the toilet or the chair, to provide more convenient options to the elderly and the disabled.

The conventional portable defecation assistive equipment is to pump air from a collecting receptacle by a pump motor to form a negative pressure condition in the collecting receptacle. A guiding cover is mounted on an opening of the collecting receptacle to attach to a buttock of a user to help defecation by the attraction force generated from the negative pressure. The conventional portable defecation equipment is mobile to help the elderly and the disabled to defecate at any place.

Though the conventional portable defecation assistive equipment is helpful, the inventor discovered some improvements to the conventional portable defecation assistive equipment after practical experiments for providing better defecation assistive equipment. So far the discovered problems are as follows:

First, with reference to FIG. 5 the conventional guiding cover 50 is thick and is made of stiff plastic, so the guiding cover 50 cannot be deformed at will to closely attach to the buttock of the user when pressed. For example, the guiding cover 50 cannot be appropriately depressed to seal the buttock of the user, and a gap is formed between the guiding cover 50 and the buttock of the user. Therefore, outer air will flow into a collecting receptacle 40 via the gap, so the negative pressure effect cannot be formed and the attraction force is reduced.

Second, the conventional guiding cover 50 has a fastening groove 51 axially concaved on an end of the guiding cover 50. The fastening groove 51 of the guiding cover 50 is disposed around an inner surface and an outer surface of an opening 41 of the collecting receptacle 40. A site of the guiding cover 50 that is mounted around the collecting receptacle 40 is thick, such that when the guiding cover 50 is attached to the buttock of the user, an inner surface and an outer surface of the fastening groove 51 are easily pulled by each other to cause forced deformation and a gap is formed between the guiding cover 50 and the collecting receptacle 40 to reduce the attraction force.

Third, the conventional defecation assistive equipment adopts a vacuum motor, but attraction force of the vacuum motor is too strong. To avoid harming the user due to the strong attraction, the conventional assistive defecation equipment is usually equipped with a pressure regulator valve to adjust the attraction force of the vacuum motor. So the conventional defecation assistive equipment has complicated structure and is complicated to use.

To overcome the shortcomings, the present invention provides a portable defecation assistive equipment to mitigate or obviate the aforementioned problems.

### SUMMARY OF THE INVENTION

The main object of the present invention is to provide a portable defecation assistive equipment to avoid the problem that the conventional defecation assistive equipment cannot attach closely to the buttock of the user and to simplify the structure.

The portable defecation assistive equipment in accordance with the present invention comprises a collecting receptacle, a guiding cover, and a pump motor. The collecting receptacle has an opening and a handle, the opening is formed on the collecting receptacle and communicates with an inner space of the collecting receptacle, and the handle is mounted on a top surface of the collecting receptacle. The guiding cover is a resilient structure, is connected to the collecting receptacle, and has a guiding hole and a wing. The guiding hole is formed through the guiding cover and communicates with the opening of the collecting receptacle. The guiding hole has two openings and a diameter of the guiding hole is gradually increased towards one of the two openings distal from the opening of the collecting receptacle. The wing is formed around an outer edge of one of the openings of the guiding hole that is bigger in diameter than the other opening of the guiding hole. The pump motor is connected to the collecting receptacle and has a gas tube, and two ends of the gas tube are respectively connected to and communicate with the pump motor and the collecting receptacle.

The portable defecation assistive equipment has a thin wing radially formed around the guiding cover. The thin wing can seal the buttock of the user to prevent gap from being formed between the guiding cover and to maintain the negative pressure condition and avoid the reduction of attraction force. In addition, the present invention adopts a pump motor having smaller attraction to avoid harming the user. So the present invention has no need to adopt an additional pressure regulator valve, the structure of the present invention is simplified, and the risk of breakdown of the pressure regulator valve is avoided.

Furthermore, the portable defecation assistive has a fastening groove radially concaved around an inner surface of the guiding hole of the guiding cover. The fastening groove communicates with an exterior environment via an end adjacent to the collecting receptacle and is disposed around and engages with the opening of the collecting receptacle. Because the fastening groove is concaved around the inner surface of the guiding hole, the fastening groove is thin and is only disposed around an outer surface of the collecting receptacle to avoid the forced deformation when the guiding cover is in use, and a gap caused by the forced deformation could be avoided to prevent the reduction of attraction force.

Other objectives, advantages and novel features of the invention will become more apparent from the following detailed description when taken in conjunction with the accompanying drawings.

### BRIEF DESCRIPTION OF THE DRAWINGS

FIG. 1 is an exploded view of a portable defecation assistive equipment in accordance with the present invention;

FIG. 2 is a perspective view of the portable defecation assistive equipment in FIG. 1;

FIG. 3 is a partially cross-sectional side view of a guiding cover and a collecting receptacle in FIG. 2;

FIG. 4 is an enlarged view of the guiding cover connecting to an opening of the collecting receptacle in FIG. 2; and

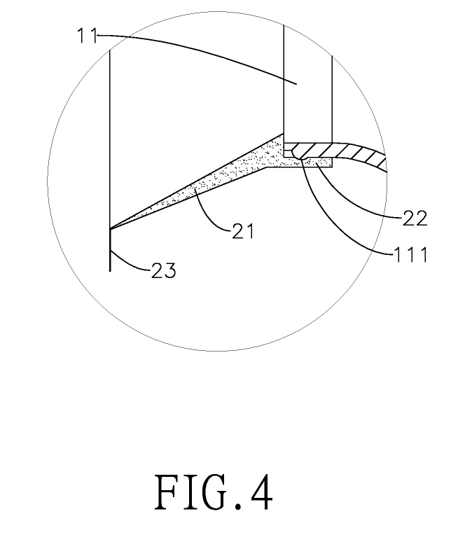

FIG. 5 is an enlarged view of a guiding cover connecting to a collecting receptacle of the conventional defecation assistive equipment.

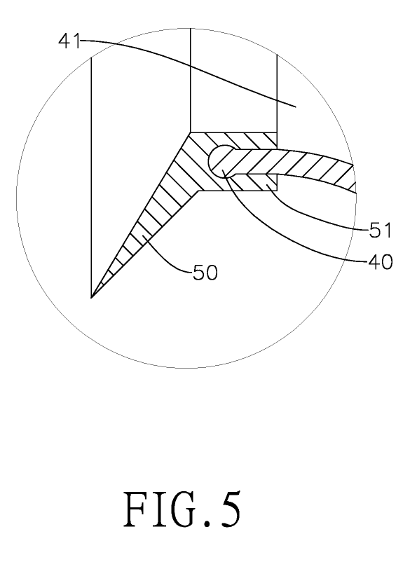

### DETAILED DESCRIPTION OF THE PREFERRED EMBODIMENTS

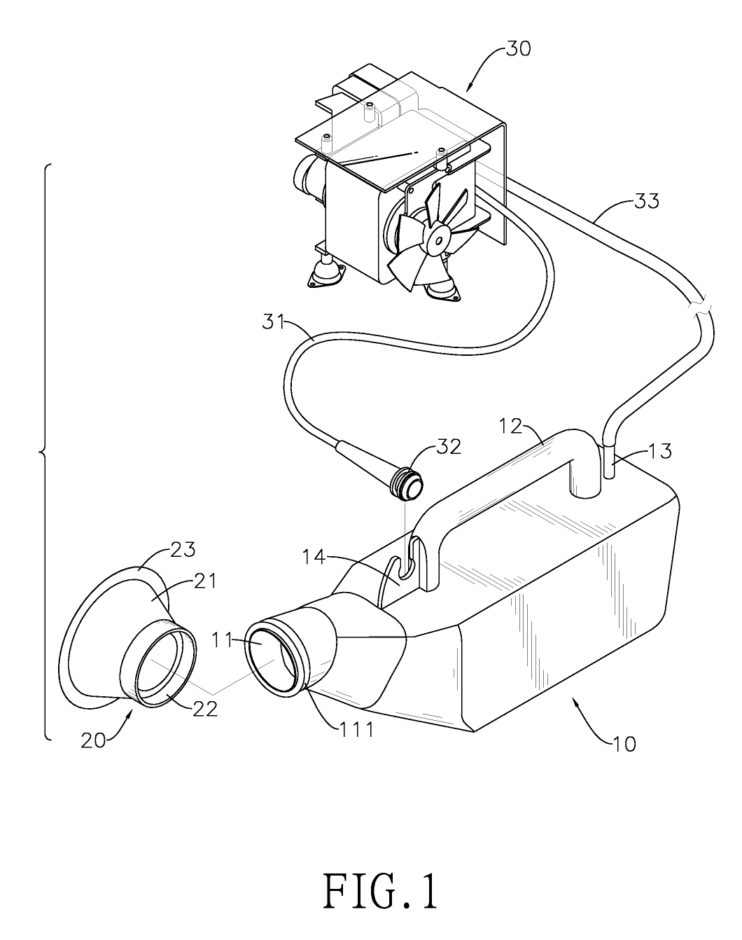

With reference to FIG. 1, a portable defecation assistive equipment in accordance with the present invention comprises a collecting receptacle 10, a guiding cover 20, and a pump motor 30.

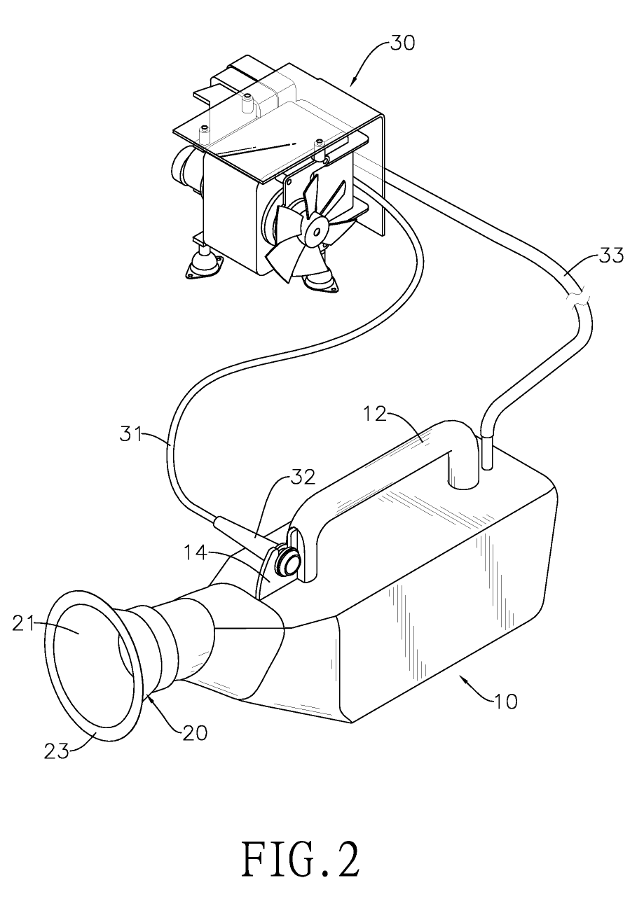

With reference to FIGS. 1 to 2, the collecting receptacle 10 is a container having an inner space and has an opening 11, a handle 12, an air suction pipe 13, and a fastening sheet 14. The opening 11 is formed on an end of the collecting receptacle 10 and communicates with the inner space of the collecting receptacle 10, and a fastening annular flange 111 extends around an outer edge of the opening 11. The handle 12 is formed on a top surface of the collecting receptacle 10. The air suction pipe 13 is mounted on the top surface of the collecting receptacle 10 and communicates with the inner space of the collecting receptacle 10. The fastening sheet 14 is formed on the top surface of the collecting receptacle 10 and is adjacent to the handle 12.

With reference to FIGS. 1 to 3, the guiding cover 20 is a resilient structure, is connected to the collecting receptacle 10, and has a guiding hole 21, a fastening groove 22, and a wing 23. The guiding hole 21 is formed through the guiding cover 20 and communicates with the opening 11 of the collecting receptacle 10. The guiding hole 21 has two openings and a diameter of the guiding hole 21 is gradually increased towards one of the two openings distal from the opening 11 of the collecting receptacle 10. The fastening groove 22 is radially concaved around an inner surface of the guiding hole 21 of the guiding cover 20 and has a slot disposed around an inner surface of the fastening groove 22. The fastening groove 22 communicates with an exterior environment via an end of the guiding cover 20 adjacent to the collecting receptacle 10 and the slot of the fastening groove 22 is disposed around and engages with the fastening annular flange 111 of the collecting receptacle 10.The wing 23 is formed around an outer edge of one of the openings of the guiding hole 21 that is bigger in diameter than the other opening of the guiding hole 21.

With reference to FIGS. 1 and 2, the pump motor 30 is connected to the collecting receptacle 10 and has a power source, an electric cable 31, a switch 32 and a gas tube 33. The power source is mounted in the pump motor 30 to provide the electricity for the operation of the pump motor 30. The electric cable 31 is electrically connected to the power source of the pump motor 30. The switch 32 is mounted on an end of the electric cable 31 to switch on or off the pump motor 30. The switch 32 is selectively fastened with the fastening sheet 14 of the collecting receptacle 10. Two ends of the gas tube 33 are respectively connected to and communicate with the pump motor 30 and the air suction pipe 13 of the collecting receptacle 10. In a preferred embodiment, the present invention adopts a pump motor 30 that has a low pumping rate to limit the pumping pressure of the pump motor 30 to be smaller than or equal to 550 mmHg.

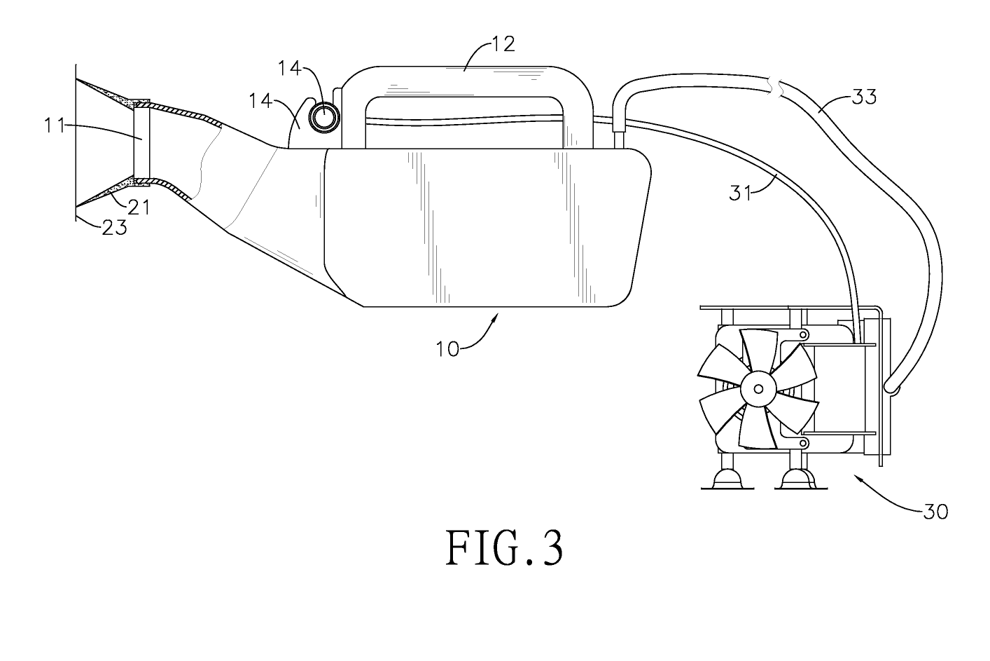

With reference to FIGS. 1 and 3, when the present invention is in use, the guiding hole 21 and the wing 23 of the guiding cover 20 are attached closely to the buttock of the user, and the switch 32 is switched on to activate the pump motor 30. The pump motor 30 starts to pump air from the inner space of the collecting receptacle 10, and the pressure in the collecting receptacle 10 is continually dropped in the pumping process. A negative pressure status is formed in the collecting receptacle 10, an attraction effect caused by the negative pressure can help the user to defecate, and excreted wastes of the user are flowed to the inner space of the collecting receptacle 10 via the guiding hole 21 of the guiding cover 20 and the opening 11.

The present invention combines the pump motor 30 and the collecting receptacle 10. The pump motor 30 pumps air from the collecting receptacle 10 that is in an air tight condition. When the pressure in the collecting receptacle 10 is smaller than the atmospheric pressure, the negative pressure status is formed in the collecting receptacle 10. The attraction caused by the negative pressure condition can help the user to defecate and the excreted wastes of the user are guided into the collecting receptacle 10 via the guiding hole 21 of the guiding cover 20 and the opening 11. The guiding cover 20 of the present invention is a resilient structure and has a wing 23 radially formed around the guiding cover 20. The wing 23 is thin and soft and can seal the buttock of the user to prevent the external air from flowing into the collecting receptacle 10, thereby maintaining the negative pressure condition and avoiding the reduction of attraction force.

Furthermore, the fastening groove 22 of the guiding cover 20 is only disposed around the outer surface of the collecting receptacle 10 to avoid the forced deformation when the guiding cover is in use, and a gap caused by the forced deformation could be avoided, thereby preventing the reduction of attraction.

In addition, the present invention adopts a pump motor having a maximum attraction of 550 mmHg to avoid harming the user. So the present invention has no need to adopt an additional pressure regulator valve, and the structure of the present invention is simplified and the risk of breakdown of the pressure regulator valve is avoided.

In another preferred embodiment, the present invention further has a storage box. The pump motor is securely mounted in the storage box. The collecting receptacle 10 is selectively mounted in the storage box. The present invention is stored in the storage box when not in use to achieve the goal of portability. For use, the user opens the storage box to retrieve the collecting receptacle 10 and make use of the present invention at any place.

In another preferred embodiment, the slot of the guiding cover could be disposed on the edge of the opening of the collecting receptacle, and the fastening annular flange is mounted on the inner surface of the fastening groove of the guiding cover. In this way, the guiding cover and the collecting receptacle could be embedded with each other.

Even though numerous characteristics and advantages of the present invention have been set forth in the foregoing description, together with details of the structure and features of the invention, the disclosure is illustrative only. Changes may be made in the details, especially in matters of shape, size, and arrangement of parts within the principles of the invention to the full extent indicated by the broad general meaning of the terms in which the appended claims are expressed.

What is claimed is:

1. A portable defecation assistive equipment comprising:
a collecting receptacle having
an opening formed on the collecting receptacle and communicating with an inner space of the collecting receptacle; and
a handle mounted on a top surface of the collecting receptacle;
a guiding cover being a resilient structure, connected to the collecting receptacle, and having
a guiding hole formed through the guiding cover and communicating with the opening of the collecting receptacle, wherein the guiding hole has two openings and a diameter of the guiding hole is gradually increased towards one of the two openings distal from the opening of the collecting receptacle;
a wing radially formed around an outer edge of one of the openings of the guiding hole that is bigger in diameter than the other opening of the guiding hole; and
a pump motor connected to the collecting receptacle and having a gas tube, and two ends of the gas tube respectively connected to and communicating with the pump motor and the collecting receptacle.

2. The portable defecation assistive equipment as claimed in claim 1, wherein the guiding cover has a fastening groove, the fastening groove is radially concaved around an inner surface of the guiding hole of the guiding cover, the fastening groove communicates with an exterior environment via an end of the guiding cover adjacent to the collecting receptacle and is disposed around and engages with the opening of the collecting receptacle.

3. The portable defecation assistive equipment as claimed in claim 2, wherein
the collecting receptacle has a fastening annular flange extending around an outer edge of the opening of the collecting receptacle; and
the fastening groove has a slot disposed around an inner surface of the fastening groove, the slot of the fastening groove is disposed around the fastening annular flange of the collecting receptacle.

4. The portable defecation assistive equipment as claimed in claim 1, wherein
the collecting receptacle further has a fastening sheet formed on the top surface of the collecting receptacle; and
the pump motor further has an electric cable and a switch, the electric cable is electrically connected to the pump motor, the switch is mounted on an end of the electric cable to switch on or off the pump motor and is selectively fastened with the fastening sheet of the collecting receptacle.

5. The portable defecation assistive equipment as claimed in claim 3, wherein
the collecting receptacle further has a fastening sheet formed on the top surface of the collecting receptacle; and
the pump motor further has an electric cable and a switch, the electric cable is electrically connected to the pump motor, the switch is mounted on an end of the electric cable to switch on or off the pump motor and is selectively fastened with the fastening sheet of the collecting receptacle.

6. The portable defecation assistive equipment as claimed in claim 1, wherein the pumping pressure of the pump motor is smaller than or equal to 550 mmHg.

7. The portable defecation assistive equipment as claimed in claim 5, wherein the pumping pressure of the pump motor is smaller than or equal to 550 mmHg.

8. The portable defecation assistive equipment as claimed in claim 1, wherein the portable defecation assistive equipment further comprising a storage box, the pump motor is securely mounted in the storage box, and the collecting receptacle is selectively mounted in the storage box.

9. The portable defecation assistive equipment as claimed in claim 7, wherein the portable defecation assistive equipment further comprising a storage box, the pump motor is securely mounted in the storage box, and the collecting receptacle is selectively mounted in the storage box.
